(19) United States
(12) Patent Application Publication
(10) Pub. No.: US 2016/0174822 A1
(43) Pub. Date: Jun. 23, 2016

(54) Multiple Viewing Element Endoscope System Having Multiple Sensor Motion Synchronization

(71) Applicant: EndoChoice, Inc., Alpharetta, GA (US)

(72) Inventors: Curtis William Stith, Santa Cruz, CA (US); Edward Andrew Jakl, Scotts Valley, CA (US)

(21) Appl. No.: 14/973,453

(22) Filed: Dec. 17, 2015

Related U.S. Application Data
(60) Provisional application No. 62/093,659, filed on Dec. 18, 2014.

(51) Int. Cl. A61B 1/00; A61B 1/045; A61B 1/05

(52) U.S. Cl. CPC A61B 1/00193; A61B 1/051; A61B 1/00009; A61B 1/0005; A61B 1/045; A61B 1/00006; A61B 1/0002; A61B 1/00055

(57) ABSTRACT

Multiple-sensor motion synchronization in a multi-viewing element endoscope system is achieved by rotating CMOS image sensors, relative to each other, and programming each CMOS image sensor to scan in specific directions. Frames collected from scans at different times are stored, processed, and displayed to form a complete image.

### CROSS-REFERENCE

The present specification relies on U.S. Patent Provisional No. 62/093,659, entitled “Multiple Viewing Element Endoscope System Having Multiple Sensor Motion Synchronization”, filed on Dec. 18, 2014 and incorporated herein by reference.

### FIELD

The present specification generally relates to multiple viewing element endoscopes utilizing complementary metal-oxide semiconductor (CMOS) image sensors. More particularly, the present specification relates to multiple viewing element endoscopes having a plurality of image sensors synchronized to capture image frames to generate a seamless image.

### BACKGROUND

Endoscopes have attained great acceptance within the medical community since they provide a means to perform procedures with minimal patient trauma while enabling the physician to view the internal anatomy of the patient. Over the years, numerous endoscopes have been developed and categorized according to specific applications, such as cystoscopy, colonoscopy, laparoscopy, upper GI endoscopy and others. Endoscopes may be inserted into the body's natural orifices or through an incision in the skin.

An endoscope is an elongated tubular shaft, rigid or flexible, having one or more video cameras or fiber optic lens assemblies at its distal end. The shaft is connected to a handle which sometimes includes an ocular device for direct viewing. Viewing is also usually possible via an external screen. Various surgical tools may be inserted through a working channel in the endoscope to perform different surgical procedures.

Endoscopes may have a front camera and a side camera to view internal organs, such as the colon, illuminators for each camera or viewing element, one or more fluid injectors to clean the camera lens(es) and sometimes also illuminator(s) and a working channel to insert surgical tools, for example, to remove polyps found in the colon. Often, endoscopes also have fluid injectors (“jet”) to clean a body cavity, such as the colon, into which they are inserted. The illuminators commonly used are fiber optics which transmit light, generated remotely, to the endoscope tip section. The use of light-emitting diodes (LEDs) for illumination is also known.

Most endoscopic viewing elements employ at least one complementary metal-oxide semiconductor (CMOS) image sensor utilizing a rolling shutter method to capture images through their viewing element. In a rolling shutter mechanism, the photodiodes (pixels) do not collect light at the same time. While all pixels in one row of the imager collect light during the same period of time, the time at which light collection starts and ends is staggered, and thus, is slightly different for each row. The top row of the imager is the first one to start collecting light and also the first row to finish collecting light, whereby this process is referred to as “readout”.

In a conventional situation, light collection starts from the top row, in a left to right direction through the row, and subsequently moves below to the next row (in a left to right direction through the row) until the process reaches the last (bottom) row. The start point and end point of the light collection for each subsequent row is slightly delayed compared to the previous row. The time delay between a row being reset and a row being read is referred to as the integration time. The integration time can be controlled by varying the amount of time between when the reset sweeps past a row and when the readout of the row takes place.

However, since there is a sequence to the integration and readout there are well-known distortions with a rolling shutter image sensor. Any moving object being captured through the rolling shutter mechanism is subject to distortion because each row of pixels “sees” the scene at a different point in time. In an example, if a target object is moving from bottom to top (or, stated differently, if the viewing element is moving top to bottom), then that image will either be compressed or elongated based on the direction of row readout.

In some multiple viewing element endoscopy systems, one viewing element may face a left direction and another may face a right direction. The viewing elements may be rotated 90 degrees clockwise or counterclockwise. If two viewing elements were rotated clockwise and were clocked (and thus integrated and readout) at the same time (for example, left to right), then during the insertion of the endoscope the viewing element on the right side may display an elongation effect because the individual row readout (left to right) would have subsequent rows “seeing” the scene physically farther away from the previous row. The viewing element on the left side may display compression because the next row of the imager would “see” the scene physically closer to the previous row. Therefore, readouts from rolling shutter CMOS image sensors may result in a compressed image from one viewing element and an elongated image from the other viewing element. Additionally, the image of an object that is moving relative to the multiple viewing elements will exhibit a discontinuity, as the image of that object moves from one viewing element to the adjacent viewing element.

Thus, there is a need for an imaging system that allows images to be viewed with continuity when multiple CMOS image sensors are utilized in an endoscope. There is also a need for an imaging system that minimizes the delay between start points and end points of light collection for each subsequent row, and therefore, minimizes image distortion, including elongation and compression.

### SUMMARY

The following embodiments and aspects thereof are described and illustrated in conjunction with systems, tools and methods, which are meant to be exemplary and illustrative, not limiting in scope.

The present specification discloses an endoscope system, comprising: at least two complementary metal-oxide semiconductor (CMOS) image sensors rotated, relative to each other, by a predetermined angle, each of said at least two CMOS image sensor having four edges, wherein each of said at least two CMOS image sensor is configured to scan a frame of an image through multiple serial columns, each scan commencing from an initial point of a column on a first edge of a sensor and ending at a final point of a column on a second opposite edge of the sensor, wherein the scan proceeds serially through each column of the sensor; and a processor connected to the multiple CMOS image sensors, the processor synchronizing the image frames scanned by the multiple image sensors by using the predetermined angle of rotation to obtain a complete image.

Optionally, the endoscope system further comprises at least one display connected to the processor for displaying the complete image, scanned by the at least two CMOS image sensors.

Optionally, each of the at least two CMOS image sensors comprises at least one register, wherein the at least one register is configured to be programmed by the processor in order to control a direction of scanning performed by one of the at least two CMOS image sensors.

The first of the at least two CMOS image sensors may be rotated, relative to the second of the at least two CMOS image sensors, by 90 degrees in a clockwise direction. The first of the at least two CMOS image sensors may be rotated, relative to the second of the at least two CMOS image sensors, by 90 degrees in a counter-clockwise direction.

The complete image may be a combination of image frames scanned by each of said at least two CMOS image sensors.

Optionally, each of the at least two CMOS image sensors is oriented in a front direction having a different forward-looking angle, relative to a direction of insertion of an insertion portion of the endoscope system inside a body cavity.

Optionally, the endoscope system further comprises a third CMOS image sensor, wherein the third CMOS image sensor is rotated, relative to one of the at least two CMOS image sensors, by 90 degrees in either a clockwise or counter-clockwise direction.

Optionally, the endoscope system further comprises a third CMOS image sensor, wherein the third CMOS image sensor is rotated, relative to one of the at least two CMOS image sensors, by 180 degrees in either a clockwise or counter-clockwise direction. The complete image may be a combination of image frames scanned by each of said at least two CMOS image sensors and the third CMOS image sensor.

The present specification also discloses an endoscope system comprising: one or more complementary metal-oxide semiconductor (CMOS) image sensors rotated a predetermined angle, each image sensor having four edges, wherein each image sensor scans a frame of an image through multiple serial columns, each scan commencing from an initial point of a column on a first edge of a sensor and ending at a final point of a column on a second opposite edge of the sensor wherein the scan proceeds serially through each column of the sensor; and a processor connected to the multiple CMOS image sensors, the processor synchronizing the image frames scanned by the multiple image sensors by using the angle of rotation to obtain the complete image.

Optionally, the endoscope system further comprises at least one display connected to the processor for displaying the complete image scanned by the multiple CMOS image sensors. Optionally, each of the multiple CMOS image sensors comprises at least one register, the register being programmable by the processor via a digital serial interface for controlling a direction of scanning performed by the sensor.

Optionally, each of the multiple CMOS image sensors is physically rotated to scan a frame of an image through multiple serial columns. Optionally, at least one of the multiple CMOS image sensors is rotated in a clockwise direction by 90 degrees or in a counter-clockwise direction by 90 degrees. The multiple CMOS image sensors may be rotated in combinations of clockwise and counter-clockwise directions, by 90 degrees. Optionally, at least one of the multiple CMOS image sensors is rotated by 180 degrees.

The complete image may be a combination of image frames scanned by each image sensor.

Optionally, each image sensor is oriented in one of a left direction, a front direction, a right direction, a top direction, and a bottom direction, relative to a direction of insertion of an insertion portion of the endoscope system inside a body cavity. Still optionally, each image sensor is oriented in a front direction having a different forward-looking angle, relative to a direction of insertion of an insertion portion of the endoscope system inside a body cavity.

The present specification also discloses a method for displaying an image obtained by using multiple complementary metal-oxide semiconductor (CMOS) image sensors in an endoscope system, each of the multiple CMOS image sensors having a top edge, a bottom edge, a left edge and a right edge, the method comprising: synchronizing each of the multiple CMOS image sensors, wherein the synchronizing comprises: setting a same first initial time (T0) of storing image frames corresponding to each of the multiple CMOS image sensors with the exception of at least one of the multiple CMOS image sensors, wherein the initial time of storing image frames of the at least one of the multiple CMOS image sensors is set to a second time (T−1 or T+1); and programming scan directions for each image sensor; scanning a frame of an image through multiple serial columns, each scan commencing from an initial point of a column on a first edge of each of the multiple CMOS image sensors and ending at a final point of a column on a second opposite edge of the multiple CMOS image sensors wherein the scan proceeds serially through each column of the sensor; storing image frames scanned by every image sensor and corresponding to the set frame time for each sensor in a frame buffer; processing the stored image frames to obtain a complete image; and displaying the complete image.

Optionally, processing the stored image frames to obtain a complete image comprises orienting the scanned image frames using a predefined orientation, wherein the complete image is a combination of the image frames scanned by each of the multiple CMOS image sensors.

The second time may be different from the first time by a time taken to scan an image frame.

Optionally, the image is a moving image or each of said multiple CMOS image sensors is in motion.

Optionally, each of the multiple CMOS image sensors is oriented in at least two different directions from a group comprising a left direction, a front direction, a right direction, a top direction, and a bottom direction, relative to a direction of insertion of the endoscope system inside a body cavity. Optionally, the orienting comprises re-mapping the complete scanned image for display.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1 shows a multiple viewing elements endoscopy system, in accordance with some embodiments of the present specification;

FIG. 2 illustrates a conventional complementary metal-oxide semiconductor (CMOS) image sensor;

FIG. 3a illustrates a sensor obtained by rotating a standard sensor in a clockwise direction, in accordance with various embodiments of the specification provided herein;

FIG. 3b illustrates a sensor obtained by rotating a standard sensor in a counter clockwise direction, in accordance with various embodiments of the specification provided herein;

FIG. 3c illustrates a plurality of images generated by modifying register settings, in accordance with various embodiments of the specification provided herein;

FIG. 4 is a flow chart illustrating a method of operation of an endoscope with multiple image sensors, in accordance with some embodiments of the present specification;

FIG. 5 illustrates three sensors, including a left-facing sensor, a front-facing sensor, and a right-facing sensor, positioned within a multiple viewing elements endoscopy system, in accordance with some embodiments of the present specification;

FIG. 6 illustrates five sensors, including a left-facing sensor, a front-facing sensor, a right-facing sensor, a top-facing sensor, and a bottom-facing sensor, within a multiple viewing elements endoscopy system, in accordance with some embodiments of the present specification;

FIG. 7 illustrates four sensors, including a left-facing sensor, a front-facing sensor, a right-facing sensor, and a top facing sensor positioned within a multiple viewing elements endoscopy system, in accordance with some embodiments of the present specification;

FIG. 8 illustrates four sensors, including a left-facing sensor, a front-facing sensor, a right-facing sensor, and a bottom facing sensor positioned within a multiple viewing elements endoscopy system, in accordance with some embodiments of the present specification;

FIG. 9 illustrates a logical, virtual, or physical circuit of a master clock, a vertical sync clock, and image sensors, in accordance with some embodiments; and

FIG. 10 illustrates a block diagram of a CMOS image sensor incorporated in an endoscopy system, in accordance with various embodiments of the specification provided herein.

### DETAILED DESCRIPTION

The present specification is directed toward multiple viewing element endoscopy systems having a plurality of image sensors wherein the image sensors are rotated 90 degrees clockwise, 90 degrees counter-clockwise, or 180 degrees relative to a conventional sensor orientation on an integrated circuit board of said endoscope. The sensors are fixed on the circuit board and not movable relative to said board after initial endoscope assembly. The scan start times of one or more of the sensors can be delayed relative to the remaining sensors. In addition, the scan direction of each sensor can be changed relative to each other sensor by programming at least one register included on the integrated circuit board. Staggering the scan start times and adjusting the scan directions of the rotated image sensors allows for the generation of a cleaner, seamless image by reducing the amount of image artifacts introduced during an image scan.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It is noted that the term “endoscope” as mentioned to herein may refer particularly to a colonoscope and a gastroscope, according to some embodiments, but is not limited only to colonoscopies and/or gastroscopies, and may include other applications such as industrial applications. The term “endoscope” may refer to any instrument used to examine the interior of a hollow organ or cavity of the body. Additionally, the term ‘viewing element’ may refer to a viewing element comprising a complementary metal-oxide semiconductor (CMOS) image sensor, and is therefore used interchangeably with the term ‘image sensor’.

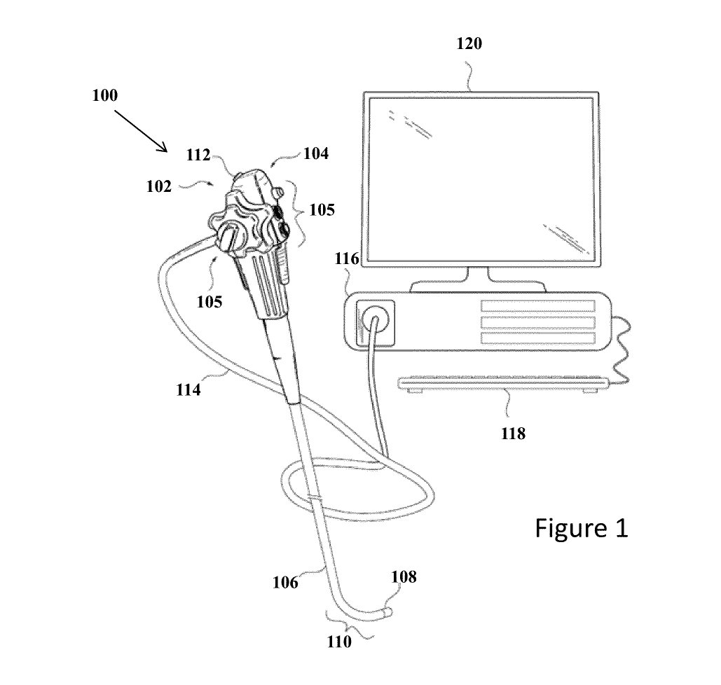

Reference is now made to FIG. 1, which shows a multiple viewing elements endoscopy system 100, in accordance with some embodiments. System 100 may include a multiple viewing elements endoscope 102. Multiple viewing elements endoscope 102 may include a handle 104 from which an elongated shaft 106 emerges. Elongated shaft 106 terminates with a tip section 108, which can be turned by way of a bending section 110, for example a vertebra mechanism. Handle 104 may be used to maneuver elongated shaft 106 within a body cavity. The handle may include one or more buttons, knobs, and/or switches 105 that control bending section 110 as well as functions such as fluid injection and suction. Handle 104 may further include a working channel opening 112 through which surgical tools may be inserted, as well as one or more side service channel openings.

A utility cable 114 may connect between handle 104 and a main control unit (MCU) 116. Utility cable 114 may include therein one or more fluid channels and one or more electrical channels. The electrical channel(s) may include at least one data cable to receive video signals from the front and side-pointing viewing elements, as well as at least one power cable to provide electrical power to the viewing elements and to the discrete illuminators. Main control unit (MCU) 116 governs a plurality of operational functionalities of the endoscope. For example, main control unit (MCU) 116 may govern power transmission to the endoscope's 102 tip section 108, such as for the tip section's viewing elements and illuminators. Main control unit (MCU) 116 may further control one or more fluid, liquid and/or suction pumps, which supply corresponding functionalities to endoscope 102. One or more input devices, such as a keyboard 118, may be connected to main control unit (MCU) 116 for the purpose of human interaction with main control unit (MCU) 116. In another configuration (not shown), an input device, such as a keyboard, may be integrated with main control unit (MCU) 116 in the same casing.

A display 120 may be connected to main control unit (MCU) 116, and configured to display images and/or video streams received from the viewing elements of multiple viewing elements endoscope 102. Display 120 may further be operative to display a user interface to allow a human operator to set various features of system 100.

Optionally, the video streams received from the different viewing elements of multiple viewing elements endoscope 102 may be displayed separately on display 120, either side-by-side or interchangeably (namely, the operator may switch between views from the different viewing elements manually). Alternatively, these video streams may be processed by main control unit (MCU) 116 to combine them into a single, panoramic video frame, based on an overlap between fields of view of the viewing elements.

In another configuration (not shown), two or more displays may be connected to main control unit (MCU) 116, each to display a video stream from a different viewing element of the multiple viewing elements endoscope.

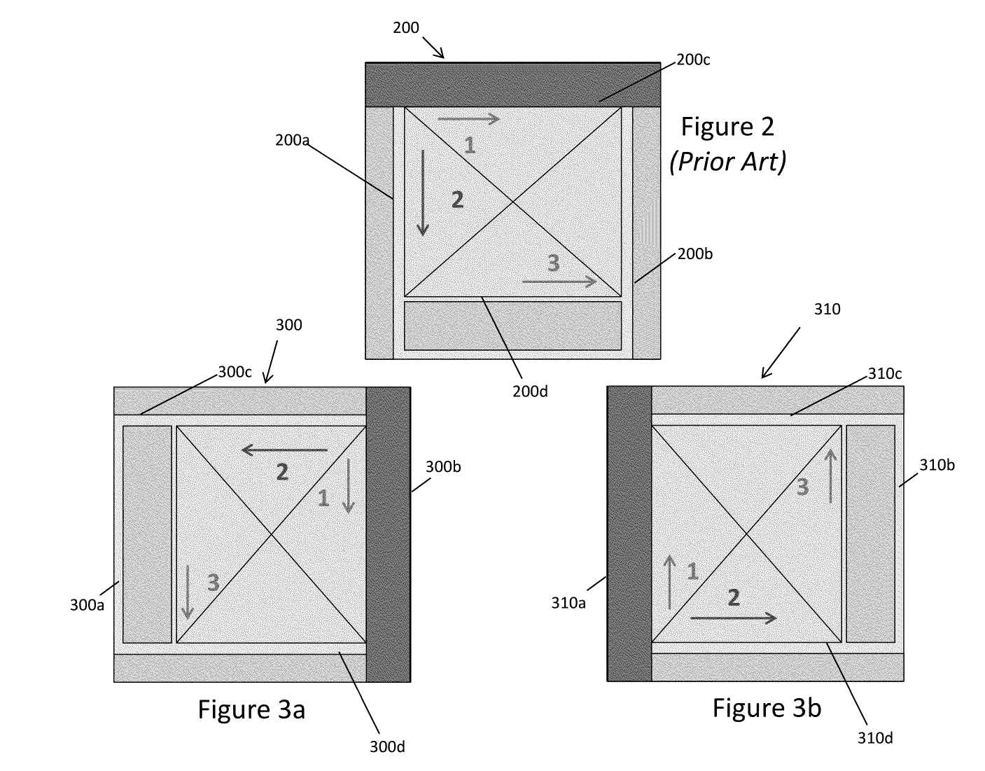

FIG. 2 illustrates a conventional CMOS image sensor 200. Arrows 1, 2, and 3 indicate the direction of a scan typically performed by sensor 200 of an image in its field of view, as also described above. The scan, which is the process of reading-out, is performed using the rolling shutter method known in the art. In this method, a still picture or a frame of a video is captured by the sensor by rapidly scanning across the field of view. Each image sensor, such as sensor 200, has four edges—a left edge 200a, a right edge 200b, a top edge 200c, and a bottom edge 200d. In FIG. 2, arrow 1 indicates a row readout start point and direction starting from the left edge 200a and along the top edge 200c. Arrow 2 indicates a direction of the progression of scans among rows. The number of rows typically ranges from 480 rows for a low-resolution sensor, up to 4000 rows for a high resolution array. Here, the scan progresses from the top row along the top edge 200c towards the bottom row along the bottom edge 200d. Arrow 3 indicates a row readout end point and direction, along the bottom edge 200d. Standard CMOS image sensors, such as sensor 200, thus scan horizontally from left to right in each row, and progress vertically down rows, starting from the top edge 200c and ending at the bottom edge 200d.

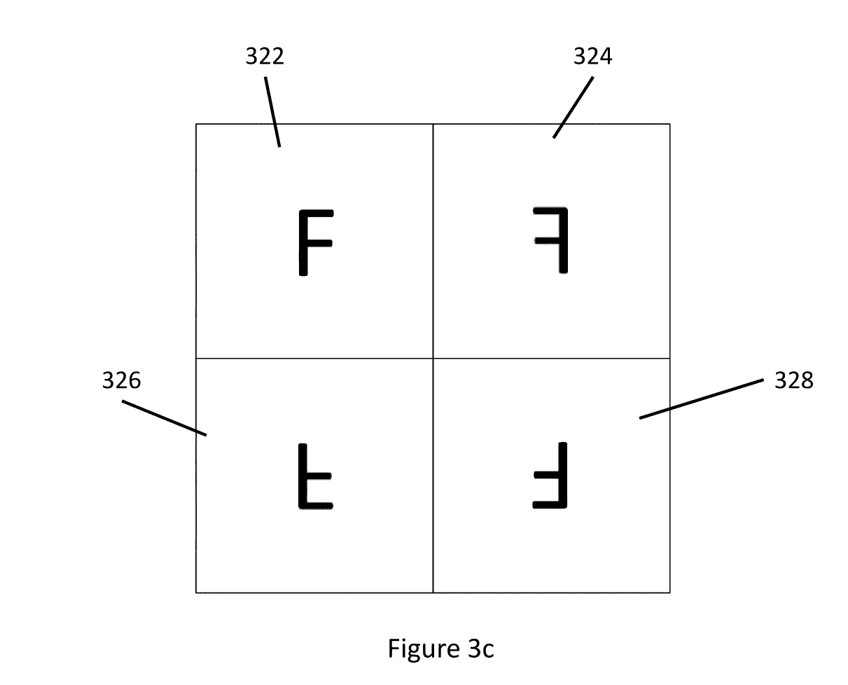

In various embodiments, a standard sensor, (such as sensor 200 depicted in FIG. 2), is rotated in a clockwise or a counterclockwise direction. FIG. 3a illustrates sensor 300 obtained by rotating a standard sensor, such as sensor 200 depicted in FIG. 2, in a clockwise direction, in accordance with an embodiment of the present specification. Sensor 300 has four edges—a left edge 300a, a right edge 300b, a top edge 300c, and a bottom edge 300d. Referring to FIGS. 2 and 3a simultaneously, left edge 300a of FIG. 3a corresponds with bottom edge 200d of FIG. 2, right edge 300b of FIG. 3a corresponds with top edge 200c of FIG. 2, top edge 300c of FIG. 3a corresponds with left edge 200a of FIG. 2, and bottom edge 300d of FIG. 3a corresponds with right edge 200b of FIG. 2. FIG. 3b illustrates sensor 310 obtained by rotating a standard sensor, such as sensor 200 depicted in FIG. 2, in a counter-clockwise direction, in accordance with an embodiment of the present specification. Sensor 310 has four edges—a left edge 310a, a right edge 310b, a top edge 310c, and a bottom edge 310d. Referring to FIGS. 2 and 3b simultaneously, left edge 310a of FIG. 3b corresponds with top edge 200c of FIG. 2, right edge 310b of FIG. 3b corresponds with bottom edge 200d of FIG. 2, top edge 310c of FIG. 3b corresponds with right edge 200b of FIG. 2, and bottom edge 310d of FIG. 3b corresponds with left edge 200a of FIG. 2. In embodiments, a CMOS sensor may be rotated 90 degrees in the clockwise direction or 90 degrees in the counter-clockwise direction. In embodiments of an endoscope system that includes multiple sensors similar to sensor 200 of FIG. 2, multiple conventional sensors may be rotated in a combination of 90 degrees clockwise and 90 degrees counter-clockwise directions. In other embodiments, the sensor(s) may be rotated by 180 degrees.

As a result of the rotation, the rows that are scanned from left edge to right edge are also rotated, and aligned from a horizontal direction to a vertical direction. Referring to FIG. 3a, the scan is now performed in columns from an initial point such as top edge 300c towards a final point such as bottom edge 300d, as compared to rows from left edge 200a towards right edge 200b in a conventional sensor 200 as shown in FIG. 2. An image that was scanned conventionally by each sensor through multiple serial rows is now scanned through multiple serial columns, in a direction between the top edge 300c and the bottom edge 300d in each column.

As a person skilled in the art would appreciate, the term ‘row’ is used to describe a horizontal direction of readout in sensor 200, while the term ‘column’ is used to describe a vertical direction of readout in sensor 300, 310. For purposes of the remaining figures and description (with the noted exception of conventional FIG. 2), arrow 1 generally indicates sensor column readout start point and direction, arrow 2 generally indicates direction of readout across multiple columns, and arrow 3 generally indicates sensor column readout end point and direction.

FIG. 3a illustrates a sensor 300 that has been rotated 90 degrees in the clockwise direction, relative to the orientation of sensor 200. In some embodiments, the rotation of sensor 300 is accomplished by physically rotating a conventional sensor (such as sensor 200 of FIG. 2) by 90 degrees clockwise during assembly of the endoscope and then fixedly mounting the sensor to an integrated circuit board of the endoscope. In other words, in some embodiments, once the endoscope has been assembled, the sensor 300 is fixed in an orientation rotated clockwise by 90 degrees relative to a standard CMOS orientation, as depicted in FIG. 2, of a prior art endoscope. The readout in sensor 300 starts from an initial edge, which may be top of right edge 300b and progresses vertically downward through a column, in a direction indicated by arrow 1. Arrow 2 indicates a readout start point and direction of progress across individual columns. In some embodiments, internal registers are used to change the direction of the scan. For example, in some embodiments, readout start point and direction of progress across columns could be from a column along the right edge 300b (right column) towards a final edge in a column along the left edge 300a (left column), or vice-versa. In various embodiments, readout progresses between two opposite edges within sensor 300. These may be done independently or concurrently which allows for up to 4 readout start points and directions. Arrow 3 indicates a column readout end point and direction.

FIG. 3b illustrates a sensor 310 that has been rotated 90 degrees in the counter clockwise direction, relative to the orientation of sensor 200. In some embodiments, the rotation of sensor 310 is accomplished by physically rotating a conventional CMOS sensor (such as sensor 200) by 90 degrees counter-clockwise during assembly of the endoscope and then fixedly mounting the sensor to an integrated circuit board of the endoscope. In other words, in some embodiments, once the endoscope has been assembled, the sensor 310 is fixed in an orientation rotated counter-clockwise by 90 degrees relative to a standard CMOS orientation, as depicted in FIG. 2, of a prior art endoscope. The readout in sensor 310 starts from an initial edge, which may be bottom of left edge 310a and progresses vertically upward through a column, in a direction indicated by arrow 1. Arrow 2 indicates a readout start point and direction of progress across individual columns. In some embodiments, internal registers are used to change the direction of the scan. For example, in some embodiments, readout start point and direction of progress across columns could be from a column along the left edge 310a (left column) towards a final edge in a column along the right edge 310b (right column), or vice-versa. In various embodiments, readout progresses between two opposite edges within sensor 310. These may be done independently or concurrently which allows for up to 4 readout start points and directions. Arrow 3 indicates a column readout end point and direction.

In other embodiments, a rotating member is attached to the sensor, such as sensors 300, 310 of FIGS. 3a and 3b respectively, in order to rotate the sensor in a desired direction. A miniature motor coupled with the rotating member enables the rotation via a controller that moves the rotating member. In these embodiments, the sensor is not fixed relative to an integrated circuit board during assembly of the endoscope and can be rotated in 90 degree increments, relative to said circuit board, during operation of the endoscope, through the use of said rotating member and miniature motor.

In some embodiments, internal registers are used to change the direction of the row scan to a column scan and vice versa, the direction of a row scan from left to right and vice versa, and/or the direction of a column scan from top to bottom and vice versa. For example, in an embodiment and referring to FIG. 3b, the readout in sensor 310 starts from an initial edge, which may be bottom of left edge 310a and progresses vertically upward through a column, in a direction indicated by arrow 1. Arrow 2 indicates a readout start point and direction of progress across individual columns. In embodiments, readout start point and direction of progress across columns could be from a bottom corner along the left edge 310a (left column) towards a column along the right edge 310b (right column), or vice-versa.

In embodiments, rotated sensors 300, 310 of FIGS. 3a and 3b respectively, are programmed by setting an appropriate internal register to change the direction of scan. A processor connected to the multiple CMOS image sensors synchronizes the scans by the multiple image sensors and orients a complete scanned image, wherein the complete scanned image is a combination of images scanned by each image sensor. In embodiments, a processor is connected to sensor 300 of FIG. 3a. The processor may synchronize the scan by sensor 300 with other sensors in the endoscope system to produce a complete scanned image. In embodiments, the complete scanned image is a combination of images scanned by each image sensor. In embodiments, programming is performed in real time when the endoscope system is initialized while powering up. In embodiments, sensor 300 contains one or more internal registers that are programmed for its operation. These registers may enable various settings such as analog gain settings, integration time, and internal voltages. The scanning direction may be programmed by setting said one or more registers in the sensor such that the scanning is performed across columns—from either left column to right column or from right column to left column, and from either the bottom corner or the top corner in either case.

In various embodiments, changing the settings of the one or more registers causes the sensor to readout the image starting from a different corner of said sensor. The sensor itself, after having been initially rotated 90 degrees clockwise or counter-clockwise, or 180 degrees, from an initial configuration during assembly, is not physically moved during scanning. Rather, in various embodiments, changing the register settings allows for scanning a mirror image, a flipped image, or a mirror and flipped image. FIG. 3c illustrates a first scanned image 322 in which none of the register settings have been changed. Changing a ‘mirror’ register setting generates image 324 while changing a ‘flip’ register setting generates image 326. Changing both ‘mirror’ and ‘flip’ register setting generates image 328. In embodiments, the registers may be set through a serial interface like I2C, SPI, or any other serial digital interface.

In embodiments, different configurations of sensors, such as sensor 300 of FIG. 3a, are combined to operate an endoscope system with multiple image sensors. Each image sensor, or viewing element, provides a readout of a scene from its field of view. Frames from multiple viewing elements are combined to generate a single image. Since the image sensors are rotated by 90 degrees, their resultant output is no longer horizontal across the row readout, from top row to bottom row, which is otherwise conventional for standard image sensors and displays. In embodiments described herein, image sensors scan between two opposite edges—for example, from top edge to bottom edge across columns, from left column to right column or from right column to left column, depending on the configuration of the viewing element. As a result, data collected from each sensor, and combined to generate a single complete image, is stored in a buffer and re-mapped to an orientation that is suitable for the monitor(s) to display the image(s). Optionally, the video streams received from the different sensors may be displayed separately on a display, either side-by-side or interchangeably (namely, the operator may switch between views from the different viewing elements manually). In another configuration, two or more displays may be utilized to display images from each sensor in the multi-viewing elements endoscope.

In embodiments of a multiple viewing element endoscope, the numbers of multiple viewing elements may vary. In some embodiments, the endoscope may include two viewing elements, three viewing elements, five viewing elements, or any other number of viewing elements appropriate for operation of the endoscope. For example, an endoscope system may include two sensors facing in different directions from a group including a right direction, a front direction, a left direction, a top direction, or a bottom direction, relative to a direction of insertion of an insertion portion of the endoscope within a body cavity. Alternatively, an endoscope system may include two sensors facing the same direction, such as the front direction, at two different viewing angles. In a multiple viewing element endoscope, at least one viewing element may be oriented to face the front direction, which is the forward direction of insertion of an insertion portion of the endoscope within a body cavity. This viewing element may be referred to as a front-facing image sensor. The other viewing element or elements may be symmetrically oriented in different and opposing directions around the front-oriented viewing element. These viewing elements may be oriented to face a direction that is 90 degrees to the left (substantially sideways) referred as left-facing sensor, 90 degrees to the right (substantially sideways in an opposite direction to the viewing element facing left) referred as right-facing sensor, 90 degrees to the top referred as top-facing sensor, or 90 degrees to the bottom (in opposite direction to the viewing element facing top) and referred as bottom-facing sensor.

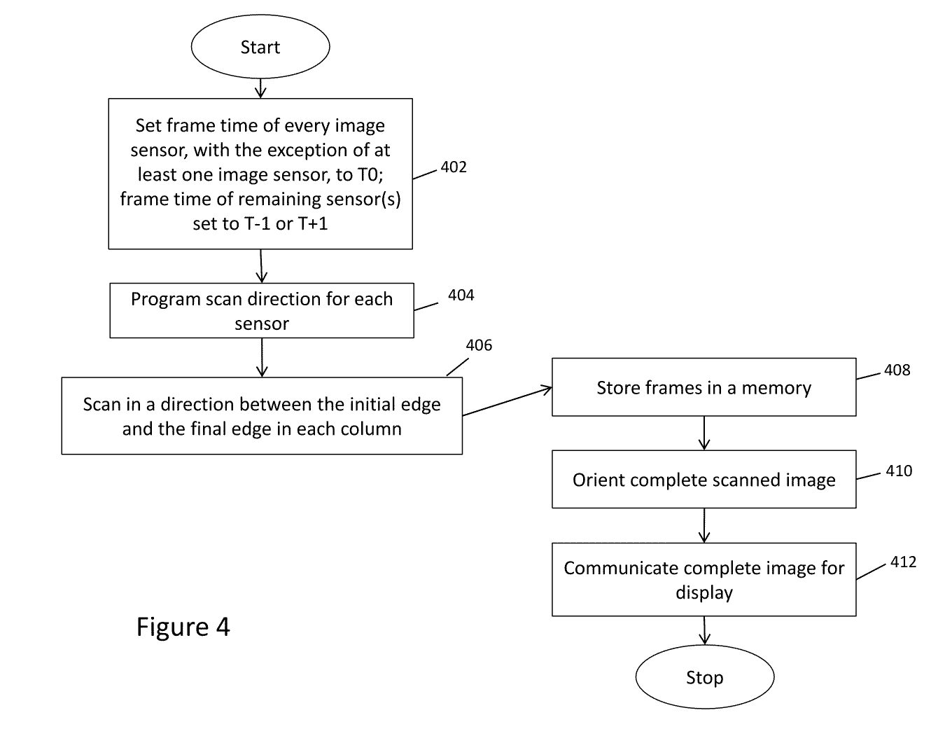

FIG. 4 is a flow chart illustrating the method of operation of an endoscope with multiple image sensors, in accordance with some embodiments. Each image sensor includes a top edge, a bottom edge, a left edge, and a right edge. Initially, each image sensor in the multiple image sensor endoscope system, is synchronized. Synchronizing involves at least steps 402 and 404. At 402, a first initial time (T0) of start of scanning a frame, also termed ‘frame time’, is set for every image sensor with the exception of at least one image sensor. For example, a front-facing image sensor and all other image sensors, except a left-facing image sensor, are set to T0. The remaining image sensors, such as the left-facing image sensor, is set to a second frame time (T+1 or T−1) to start scanning at that time. T+1 may indicate a frame time that starts at a time corresponding to the time of finishing scan of frame that started at time T0. Similarly, time T−1 may indicate a frame time that starts at a time preceding start time of T0 by the amount of time to scan one frame. Thus, at least one image sensor is set to start scanning at a time difference of one frame before or after start of scanning by all other image sensors.

At 404, a scan direction of each image sensor is programmed. The direction of scan may be programmed by setting an appropriate internal register(s) to change direction of scan of an image sensor. In one embodiment, programming may determine a starting point of the scan. In embodiments, the programming is performed in real time when the endoscope system is initialized while powering up. In embodiments, each image sensor contains multiple internal registers that are programmed for its operation. These registers may enable various settings such as analog gain settings, integration time, and internal voltages. The scanning direction may be programmed by setting at least one register (single or multiple registers) in the sensor, such that the scanning is performed across columns—from either left column to right column or from right column to left column. The column from where the scanning starts may be termed an initial edge, and the column where the scanning is performed last may be termed a final edge. In embodiments, the registers may be set through a serial interface like I2C, SPI, or any other serial digital interface.

At 406, scanning starts based on the synchronizing by each image sensor. As described with reference to FIG. 3, an image is scanned by each sensor through multiple serial columns, in a direction between the initial edge and the final edge in each column. Programming in the previous step decides the direction of scan in each image sensor. Each image sensor has four edges—a left edge, a right edge, a top edge, and a bottom edge. In some embodiments, as shall be discussed with reference to subsequent figures, the image sensor oriented to face the left direction is set to start scanning at frame time T+1 or T−1, relative to other sensors including the front-facing sensor. This is so that if the left and front facing sensors start scanning from their right edges, the right edge column on the left-facing sensor is clocked (integrated or exposed) at the same time as the left side edge column of the front-facing sensor. It takes a full frame time to read from the first column to the last column. So, if the left-facing sensor were not offset by one frame then its edge column would be one full frame time away from the edge column of the front-facing sensor. Thus, lack of the offset could result in a discontinuity when sensors are in motion while capturing a scene.

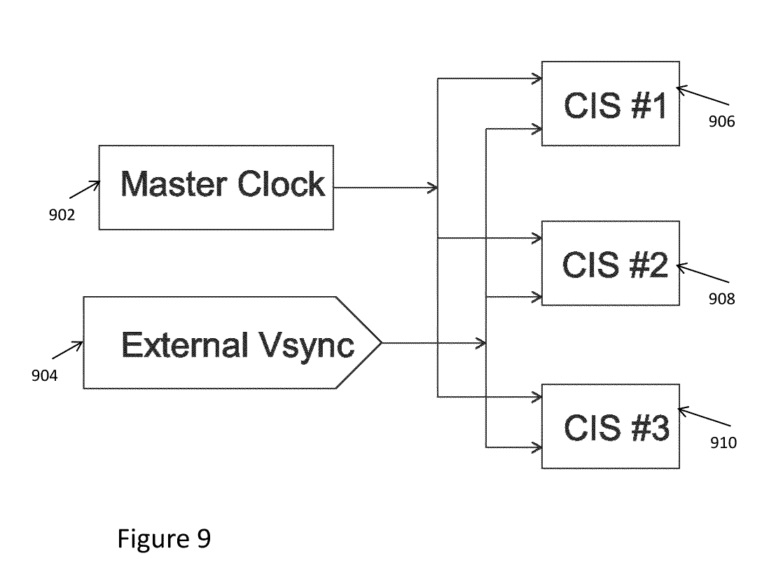

In embodiments, synchronizing the time of start of scan by the image sensors requires a master clock and vertical sync clock for operation. FIG. 9 illustrates an exemplary network of a master clock 902, a vertical sync clock 904, and image sensors 906, 908, and 910, in accordance with some embodiments. The master clock 902 directs, from a timing perspective, the integrated circuit board of the endoscope. Vertical sync 904 may be common to all image sensors 906, 908, and 910 in order to synchronize the start of frame. The vertical sync 904 is external to, and independent from, the master clock 902. The vertical sync 904 generates an external signal which instructs each sensor to start (scan) a frame. The vertical sync 904 ensures that all sensors set to scan at the same time will be synchronized and start their scans simultaneously. If the multiple image sensors 906, 908, and 910 were not synchronized, they would be free-running and the images captured by them would not be synchronized leading to motion discontinuities between scenes from each sensor. The vertical sync 904 determines the frame rate which, in various embodiments, is set within a range of 1 frame per second to 300 frames per second. In one embodiment, the frame rate is set to 30 frames per second. In another embodiment, the frame rate is set to 15 frames per second. While FIG. 9 illustrates an embodiment with three sensors, it should be understood that in alternative embodiments, a similar network may be utilized for any other numbers of sensors.

Referring back to the method described in FIG. 4, at 408, scanned frames corresponding to the set frame time for each sensor are stored in a memory. The sensors may not have on-board memory and may be unable to store any frame data. Therefore, in embodiments, a frame buffer is incorporated in the endoscope system to store frame from time T−1 from the left-facing sensor while waiting for frames from time T0 from the other. Alternatively, in embodiments, the frame buffer stores frames from time T0 from all other sensors while waiting for frame at time T+1 from the left-facing sensor.

Optionally, at 410, frames scanned by each sensor and stored in the memory are combined and oriented to form a complete scanned image. In embodiments described herein, image sensors scan left to right or right to left (horizontally across columns) depending on the configuration of the viewing element. As a result, data collected from each sensor may be combined to generate a single complete image, which is stored in a buffer and re-mapped to an orientation that is suitable for the monitor(s). At 412, the appropriately oriented image is communicated to display on a monitor. Optionally, the video streams received from different sensors may be displayed separately on a display, either side-by-side or interchangeably (namely, the operator may switch between views from the different viewing elements manually). In another configuration, two or more displays may be utilized to display images from each sensor in the multi-viewing elements endoscope.

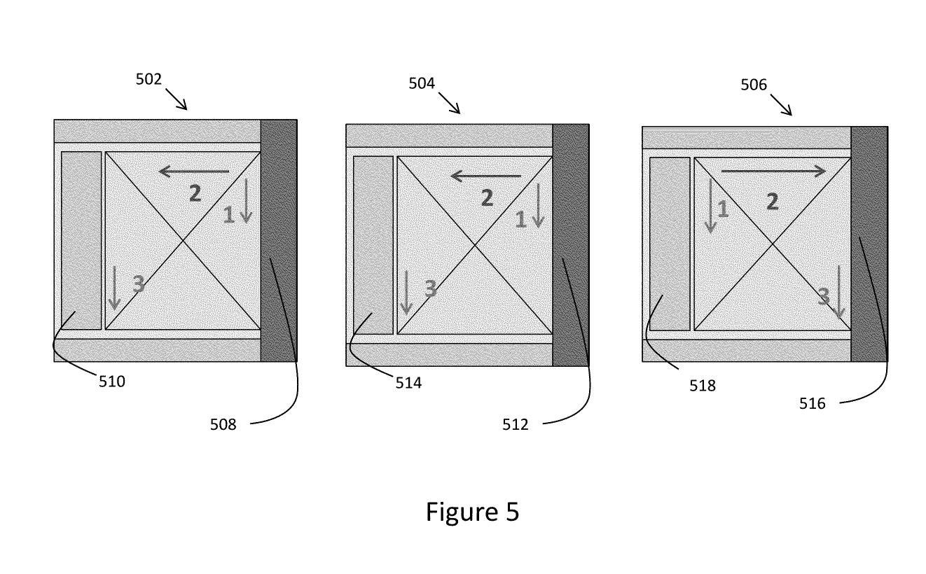

The method described in context of flow chart of FIG. 4 may apply to various multiple viewing element endoscope configurations, such as, but not limited to, two viewing element, three viewing element, four viewing element, or five viewing element endoscopes. FIG. 5 illustrates three-sensor motion synchronization utilizing embodiments of the method described with FIG. 4, in accordance with some embodiments. Referring to FIG. 5, three sensors include a left-facing sensor 502, a front-facing sensor 504, and a right-facing sensor 506 that are placed in a multiple viewing element endoscope system. Sensors 502, 504, and 506 are rotated by 90 degrees in the clockwise direction, relative to a standard sensor 200 of FIG. 2. Each image sensor 502, 504, and 506, has four edges including a top edge, a bottom edge, a right edge, and a left edge. Direction of scan may be set during initialization, for each image sensor 502, 504, and 506. In embodiments, a synchronization clock, such as vertical sync clock 904 of FIG. 9, ensures that all the sensors are initialized at the same time. In embodiments, the scan direction is set via a serial command interface. The scan progresses through multiple serial columns, starting from the right edge serially towards the left edge, and from the top edge towards the bottom edge in each column. In embodiments, left-facing sensor 502 starts scanning from top of its right edge 508, and progresses towards its left edge 510. Front-facing sensor 504 starts scanning from top of its right edge 512, and progresses towards its left edge 514. Right-facing sensor 506 starts scanning from top of its left edge 518, and progresses towards its right edge 516. Right-facing sensor 506 may be a mirror embodiment of left-facing sensor 502, such that its scan direction may start from the left edge 518 serially towards the right edge 516, and from the top edge towards the bottom edge in each column.

Referring to FIG. 4 in context of this embodiment, at 402 and 404, image sensors 502, 504, and 506 are synchronized. During synchronization, the frame time for the image sensors 504 and 506, oriented in the front direction and the right direction, is set to a first time (T0). Frame for sensor 502 is set to a second time (T−1 or T+1), which is one frame prior to or later than the frames from sensors 504 and 506. Scan directions for each image sensor, as described above, are also programmed during synchronization.

At 406, scanning of an image is performed by each image sensor, and is based on the synchronizing. At 408, frames from time T0 from sensor 504 and 506 are combined and sent to the display. In embodiments, the sensors may not have on-board memory and may be unable to store any frame data. Therefore, a frame buffer may be incorporated in the endoscope system to store frame from time T−1 from sensor 502 while waiting for frames from time T0 from sensors 504 and 506. Alternatively, in embodiments, the frame buffer stores frames from time T0 from sensors 504 and 506 while waiting for frame at time T+1 from sensor 502. As described earlier, the second time may be different from the first time by a time taken to scan the frame.

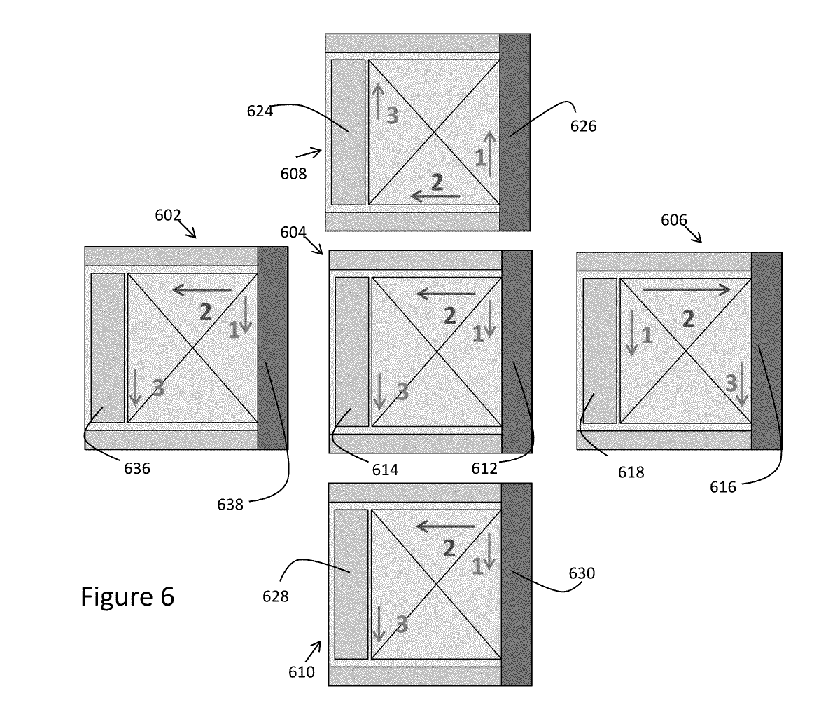

Referring to FIG. 6, five sensors include a left-facing sensor 602, a front-facing sensor 604, a right-facing sensor 606, a top-facing sensor 608, and a bottom-facing sensor 610, are placed in a multiple viewing element endoscope system, in accordance with some embodiments. FIG. 6 includes five sensors to generate a larger, more panoramic image when compared to an image generated by the three sensors of FIG. 5. Sensors 602, 604, 606, 608, and 610 are rotated by 90 degrees in the clockwise direction, relative to a standard sensor 200 of FIG. 2. Further, in embodiments, the scan direction of sensors 602, 604, and 610 is such that scan starts from their top right edges and progresses serially towards their left edges. However, the scan directions of sensors 606 and 608 are different, and are described subsequently. Each image sensor 602, 604, 606, 608, and 610, has four edges including a top edge, a bottom edge, a right edge, and a left edge.

The scan direction may be set during initialization, for each image sensor 602, 604, 606, 608, and 610. The time domain of each image sensor 602, 604, 606, 608, and 610 is set to minimize motion artifacts in the generated image. For example, the time domain for sensors 602 and 604 may be set to T0 while the time domain for sensor 606 is set to T+1 or T−1 to minimize the creation of motion artifacts as the endoscope is moved horizontally. In embodiments, a synchronization clock, such as vertical sync clock 904 of FIG. 9, ensures that all the sensors set to the same domain times are initialized at the same time. In embodiments, the scan direction is set via a serial command interface. The scan progresses through multiple serial columns, starting from the right edge serially towards the left edge or from the left edge to the right edge, and from the top edge towards the bottom edge, or the bottom edge towards the top edge, in each column. In embodiments, left-facing sensor 602 starts scanning from top of its right edge 638, and progresses towards its left edge 636. Front-facing sensor 604 starts scanning from top of its right edge 612, and progresses towards its left edge 614. Right-facing sensor 606 may be a mirror embodiment of left-facing sensor 602, such that its scan direction may start from top of its left edge 618, and progresses serially towards its right edge 616, and from the top edge towards the bottom edge in each column. In embodiments, top-facing sensor 608 is a vertically flipped embodiment of left-facing sensor 602. The scan direction of sensor 608 may start from bottom of its right edge 626, and progresses serially towards its left edge 624, and from the bottom edge towards the top edge in each column. The flipped embodiment may be obtained by clocking sensor 608 bottom to top instead of top to bottom. Bottom-facing sensor 610 starts scanning from top of its right edge 630, and progresses towards its left edge 628.

Referring to FIG. 4 in context of this embodiment, at 402 and 404, image sensors 602, 604, 606, 608, and 610 are synchronized. In an exemplary embodiment, during synchronization, the frame time for the image sensors 604, 606, 608, and 610, is set to a first time (T0). Frame for sensor 602 is set to a second time (T−1 or T+1), which is one frame prior to or later than the frames from sensors 604, 606, 608, and 610. In other embodiments, the frame times for the various image sensors 602, 604, 606, 608, and 610 are set to T0, T−1, or T+1 to minimize motion artifacts introduced during movement of the endoscope. In one embodiment, T−1 starts a scan 30 msec before a scan set to T0 and T+1 starts a scan 30 msec after a scan set to T0. Scan directions for each image sensor, as described above, are also programmed during synchronization.

At 406, scanning of an image is performed by each image sensor, and is based on the synchronizing. At 408, frames from time T0 from sensors 604, 606, 608, and 610 are combined and sent to the display. In embodiments, a frame buffer is incorporated in the endoscope system to store frame from time T−1 from sensor 602 while waiting for frames from time T0 from sensors 604, 606, 608, and 610. Alternatively, in embodiments, the frame buffer stores frames from time T0 from sensors 604, 606, 608, and 610 while waiting for frame at time T+1 from sensor 602. As described earlier, the second time may be different from the first time by a time taken to scan the frame.

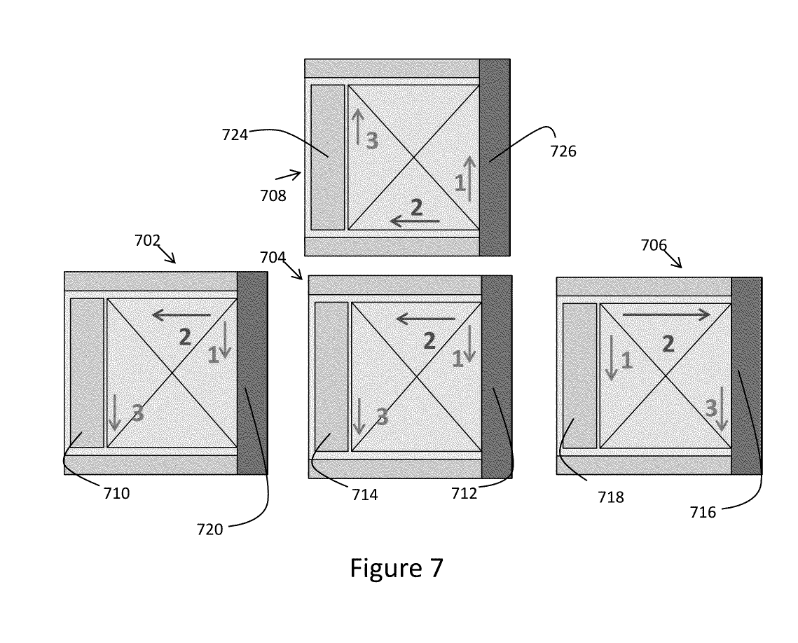

FIG. 7 illustrates yet another embodiment where four sensors include a left-facing sensor 702, a front-facing sensor 704, a right-facing sensor 706, and a top-facing sensor 708 that are placed in a multiple viewing element endoscope system, in accordance with some embodiments. Sensors 702, 704, 706, and 708 are rotated by 90 degrees in the clockwise direction, relative to a standard sensor 200. Each image sensor 702, 704, 706, and 708, has four edges including a top edge, a bottom edge, a right edge, and a left edge. Direction of scan may be set during initialization, for each image sensor 702, 704, 706, and 708. In embodiments, a synchronization clock, such as vertical sync clock 904 of FIG. 9, ensures that all the sensors are initialized at the same time. In embodiments, the scan direction is set via a serial command interface. The scan progresses through multiple serial columns, starting from the right edge serially towards the left edge or from the left edge to the right edge, and from the top edge towards the bottom edge, or the bottom edge towards the top edge, in each column. In embodiments, left-facing sensor 702 starts scanning from top of its right edge 720, and progresses towards its left edge 710. Front-facing sensor 704 starts scanning from top of its right edge 712, and progresses towards its left edge 714. Right-facing sensor 706 may be a mirror embodiment of left-facing sensor 702, such that its scan direction may start from top of its left edge 718, and progresses serially towards its right edge 716, and from the top edge towards the bottom edge in each column. In embodiments, top-facing sensor 708 is a vertically flipped embodiment of left-facing sensor 702. The scan direction of sensor 708 may start from bottom of its right edge 726, and progresses serially towards its left edge 724, and from the bottom edge towards the top edge in each column. The flipped embodiment may be obtained by clocking sensor 708 bottom to top instead of top to bottom.

Referring to FIG. 4 in context of this embodiment, at 402 and 404, image sensors 702, 704, 706, and 708 are synchronized. In one embodiment, during synchronization, the frame time for the image sensors 704, 706, and 708, is set to a first time (T0). Frame for sensor 702 is set to a second time (T−1 or T+1), which is one frame prior to or later than the frames from sensors 704, 706, and 708. Scan directions for each image sensor, as described above, are also programmed during synchronization.

At 406, scanning of an image is performed by each image sensor, and is based on the synchronizing. At 408, frames from time T0 from sensors 704, 706, and 708 are combined and sent to the display. In embodiments, a frame buffer is incorporated in the endoscope system to store frame from time T−1 from sensor 702 while waiting for frames from time T0 from sensors 704, 706, and 708. Alternatively, in embodiments, the frame buffer stores frames from time T0 from sensors 704, 706, and 708 while waiting for frame at time T+1 from sensor 702. As described earlier, the second time may be different from the first time by a time taken to scan the frame.

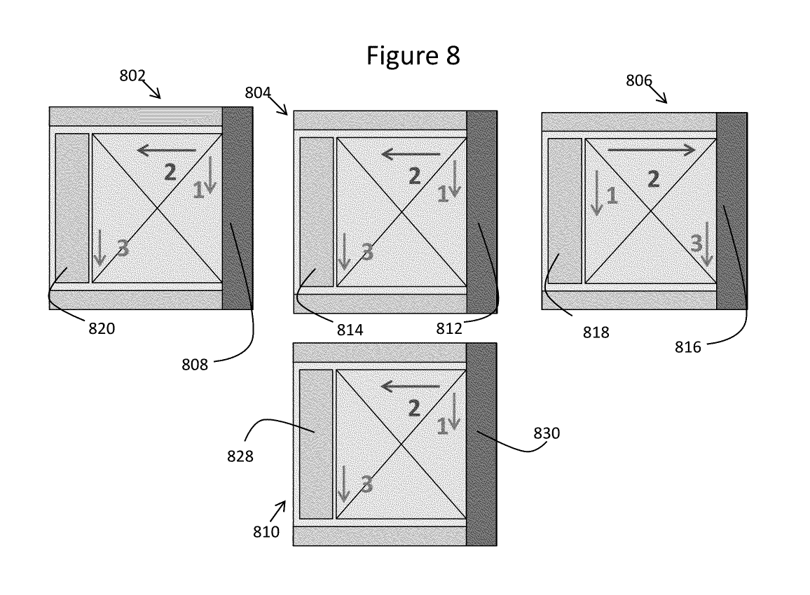

FIG. 8 illustrates still another embodiment where four sensors include a left-facing sensor 802, a front-facing sensor 804, a right-facing sensor 806, and a bottom-facing sensor 810 that are placed in a multiple viewing element endoscope system, in accordance with some embodiments. Sensors 802, 804, 806, and 810 are rotated by 90 degrees in the clockwise direction, relative to a standard sensor 200. Each image sensor 802, 804, 806, and 810, has four edges including a top edge, a bottom edge, a right edge, and a left edge. Direction of scan may be set during initialization, for each image sensor 802, 804, 806, and 810. In embodiments, a frame synchronization clock, such as vertical sync clock 904 of FIG. 9, ensures that the start of frame for all sensors occurs at the same time. In embodiments, the scan direction is set via a serial command interface. The scan progresses through multiple serial columns, starting from the right edge serially towards the left edge or from the left edge towards the right edge, and from the top edge towards the bottom edge in each column. In embodiments, left-facing sensor 802 starts scanning from top of its right edge 808, and progresses towards its left edge 820. Front-facing sensor 804 starts scanning from top of its right edge 812, and progresses towards its left edge 814. Right-facing sensor 806 may be a mirror embodiment of left-facing sensor 802, such that its scan direction may start from top of its left edge 818, and progresses serially towards its right edge 816, and from the top edge towards the bottom edge in each column. Bottom-facing sensor 810 starts scanning from top of its right edge 830, and progresses towards its left edge 828.

Referring to FIG. 4 in context of this embodiment, at 402 and 404, image sensors 802, 804, 806, and 810 are synchronized. In an embodiment, during synchronization, the frame time for the image sensors 804, 806, and 810, is set to a first time (T0). Frame for sensor 802 is set to a second time (T−1 or T+1), which is one frame prior to or later than the frames from sensors 804, 806, and 810. Scan directions for each image sensor, as described above, are also programmed during synchronization.

At 406, scanning of an image is performed by each image sensor, and is based on the synchronizing. At 408, frames from time T0 from sensors 804, 806, and 810 are combined and sent to the display. In embodiments, a frame buffer is incorporated in the endoscope system to store frame from time T−1 from sensor 802 while waiting for frames from time T0 from sensors 804, 806, and 810. Alternatively, in embodiments, the frame buffer stores frames from time T0 from sensors 804, 806, and 810 while waiting for frame at time T+1 from sensor 802. As described earlier, the second time may be different from the first time by a time taken to scan the frame.

In alternative embodiments, different physical rotations and configurations of image sensors may be used to scan vertically from different edges. Embodiments of the specification enable overcoming discontinuity in combining moving images captured by multiple rolling shutter CMOS image sensors used in endoscopes. Image compression or elongation is synchronized for each image sensor as the endoscope moves in a forward or a backward direction.

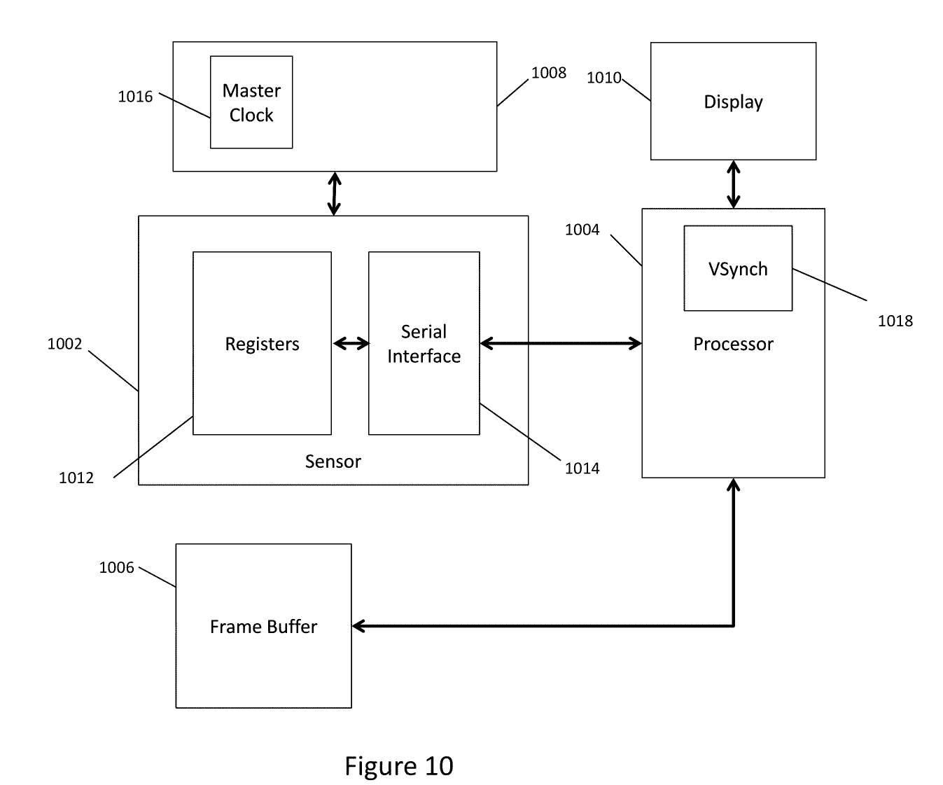

FIG. 10 illustrates a block diagram of a CMOS image sensor incorporated in an endoscopy system, in accordance with various embodiments of the specification provided herein. FIG. 10 shows a sensor 1002 coupled with a processor 1004, a frame buffer 1006 and a clock 1008. Only one sensor 1002 is shown in the figure for ease of illustration. It will be readily appreciated by persons of skill in the art that multiple such sensors are coupled with the processor 1004 and the frame buffer 1006 in an endoscopy system, wherein the processor obtains a complete image by using the image frames scanned by each sensor some of which frames are temporarily stored in the frame buffer 1006. The complete image is then displayed on a suitable display 1010 coupled with the processor 1004.

Sensor 1002 comprises a plurality of internal registers 1012 which may be used for programming the sensor's 1002 direction of scanning corresponding to a conventional sensor such as sensor 200 shown in FIG. 2. In embodiments, sensor 1002 is physically rotated by 90 degrees and programmed by setting an appropriate internal register to change the direction of scan. The processor 1004 synchronizes the scanned image frames obtained from the sensor 1002 and orients a complete scanned image, wherein the complete scanned image may be a combination of images scanned by each of multiple image sensors employed in the endoscopy system.

In embodiments, programming of the registers 1012 is performed in real time when the endoscope system is initialized while powering up. In embodiments, the multiple internal registers 1012 are programmed for aiding operation of the sensor 1002. These registers 1012 may enable various settings such as analog gain settings, integration time, internal voltages, among other settings. The scanning direction may be programmed by setting a single register in the sensor 1002, such that the scanning is performed across columns—from either left column to right column or from right column to left column, and from either the bottom corner or the top corner in either case. In embodiments, the registers 1012 may be set through a serial interface 1014 like I2C, SPI, or any other serial digital interface.

In various embodiments, scanned image frames corresponding to the set frame time for each sensor are stored in a memory as explained in conjunction with step 408 of FIG. 4. In an embodiment, the sensors are not provided with on-board memory, hence being unable to store any scanned image frame data. Therefore, in some embodiments, a frame buffer 1006 is incorporated in the endoscope system to store frame from time T−1 from, for example, at least one left-facing sensor while waiting for frames from time T0 from the other sensors. Alternatively, in other embodiments, the frame buffer 1006 stores frames from time T+1 from all other sensors while waiting for frame at time T0 from a left-facing sensor.

In embodiments, synchronizing the time of start of scan by the image sensor 1002 and other sensors (not shown in FIG. 10) coupled with processor 1004 requires a master clock 1016 and external vertical sync clock 1018 for operation as explained in conjunction with FIG. 9.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An endoscope system, comprising:
at least two complementary metal-oxide semiconductor (CMOS) image sensors rotated, relative to each other, by a predetermined angle, each of said at least two CMOS image sensor having four edges, wherein each of said at least two CMOS image sensor is configured to scan a frame of an image through multiple serial columns, each scan commencing from an initial point of a column on a first edge of a sensor and ending at a final point of a column on a second opposite edge of the sensor, wherein the scan proceeds serially through each column of the sensor; and
a processor connected to the multiple CMOS image sensors, the processor synchronizing the image frames scanned by the multiple image sensors by using the predetermined angle of rotation to obtain a complete image.

2. The endoscope system of claim 1 further comprising at least one display connected to the processor for displaying the complete image, scanned by the at least two CMOS image sensors.

3. The endoscope system of claim 1 wherein each of the at least two CMOS image sensors comprise at least one register, wherein the at least one register is configured to be programmed by the processor in order to control a direction of scanning performed by one of the at least two CMOS image sensors.

4. The endoscope system of claim 1, wherein a first of the at least two CMOS image sensors is rotated, relative to the second of the at least two CMOS image sensors, by 90 degrees in a clockwise direction.

5. The endoscope system of claim 1, wherein a first of the at least two CMOS image sensors is rotated, relative to the second of the at least two CMOS image sensors, by 90 degrees in a counter-clockwise direction.

6. The endoscope system of claim 1, further comprising a third CMOS image sensor, wherein the third CMOS image sensor is rotated, relative to one of the at least two CMOS image sensors, by 90 degrees in either a clockwise or counter-clockwise direction.

7. The endoscope system of claim 1, further comprising a third CMOS image sensor, wherein the third CMOS image sensor is rotated, relative to one of the at least two CMOS image sensors, by 180 degrees in either a clockwise or counter-clockwise direction.

8. The endoscope system of claim 7, wherein the complete image is a combination of image frames scanned by each of said at least two CMOS image sensors and the third CMOS image sensor.

9. The endoscope system of claim 1, wherein the complete image is a combination of image frames scanned by each of said at least two CMOS image sensors.

10. The endoscope system of claim 1, wherein each of the at least two CMOS image sensors is oriented in a front direction having a different forward-looking angle, relative to a direction of insertion of an insertion portion of the endoscope system inside a body cavity.

11. A method for displaying an image obtained by using multiple complementary metal-oxide semiconductor (CMOS) image sensors in an endoscope system, each of the multiple CMOS image sensors having a top edge, a bottom edge, a left edge and a right edge, the method comprising:
synchronizing each of the multiple CMOS image sensors, wherein the synchronizing comprises:
setting a same first initial time (T0) of storing image frames corresponding to each of the multiple CMOS image sensors with the exception of at least one of the multiple CMOS image sensors, wherein the initial time of storing image frames of the at least one of the multiple CMOS image sensors is set to a second time (T−1 or T+1);
programming scan directions for each image sensor;
scanning a frame of an image through multiple serial columns, each scan commencing from an initial point of a column on a first edge of each of the multiple CMOS image sensors and ending at a final point of a column on a second opposite edge of the multiple CMOS image sensors, wherein the scan proceeds serially through each column of the sensor;
storing image frames scanned by every image sensor and corresponding to the set frame time for each sensor in a frame buffer;
processing the stored image frames to obtain a complete image; and
displaying the complete image.

12. The method of claim 11 wherein processing the stored image frames to obtain a complete image comprises orienting the scanned image frames using a predefined orientation, wherein the complete image is a combination of the image frames scanned by each of the multiple CMOS image sensors.

13. The method of claim 11 wherein the second time is different from the first time by a time taken to scan an image frame.

14. The method of claim 11, comprising scanning by each of the multiple CMOS image sensors of an image, wherein the image is a moving image.

15. The method of claim 11, comprising scanning by each image sensor of the multiple CMOS image sensors, wherein each of said multiple CMOS image sensors is in motion.

16. The method of claim 11, comprising scanning by each of the multiple CMOS image sensors, wherein each of the multiple CMOS image sensors is oriented in at least two different directions from a group comprising a left direction, a front direction, a right direction, a top direction, and a bottom direction, relative to a direction of insertion of the endoscope system inside a body cavity.

17. The method of claim 12, wherein orienting comprises re-mapping the complete scanned image for display.
